(19) United States
(12) Patent Application Publication
(10) Pub. No.: US 2022/0059197 A1
(43) Pub. Date: Feb. 24, 2022

(54) SYSTEM AND METHOD FOR MONITORING COMPLIANCE AND PARTICIPANT SAFETY FOR CLINICAL TRIALS

(71) Applicant: Halo Health Systems, Inc., Plano, TX (US)

(72) Inventors: Vrunjal Mehta, Frisco, TX (US); Aneri Mehta, Frisco, TX (US)

(73) Assignee: Halo Health Systems, Inc

(21) Appl. No.: 17/402,746

(22) Filed: Aug. 16, 2021

Related U.S. Application Data
(60) Provisional application No. 63/067,372, filed on Aug. 19, 2020.

(51) Int. Cl. G16H 10/20

(52) U.S. Cl. CPC G16H 10/20

(57) ABSTRACT

A system and method for monitoring compliance and participant safety for one or more clinical trials in a computing environment is disclosed. The method includes performing clinical trials on a participant for at least one investigational product and receiving health data of the participant from one or more data sources. The method further includes monitoring health condition of the participant by analyzing the health data during one or more clinical trial protocols and determining whether the received health data meets a safe health condition level using a health condition-based AI model. The method includes predicting adverse effect level of the one or more clinical trials on the participant if the health data fails to meet the safe health condition level and performing one or more health tasks based on the predicted adverse effect level and with respect to the one or more clinical trial protocols.

### EARLIEST PRIORITY DATE

This Application claims priority from a Provisional patent application filed in the United States of America having Patent Application No. 63/067,372, filed on Aug. 19, 2020, and titled “SYSTEM AND METHOD TO MONITOR COMPLIANCE AND PATIENT SAFETY FOR CLINICAL TRIALS”.

### FIELD OF INVENTION

Embodiments of the present disclosure relate to a monitoring system for clinical trials and more particularly relates to a system and a method for monitoring compliance and participant safety for one or more clinical trials in a computing environment.

### BACKGROUND

Typically, clinical trials are performed to test investigational products such as pharmaceutical product, diagnostic test and medical devices on human beings for the purpose of validating the investigational product's safety and effectiveness. With the advancements in technology, traditional clinical trial models are exchanged with modem clinical trial models such as virtual, hybrid and decentralized models to reduce cost and improve efficiency of the traditional clinical trial models. The modem clinical trial models replace traditional research site visits with a virtual or remote visit for remote collection of data by participant or an at-home visit by a qualified clinician.

Further, compliance is an essential part of every clinical trial as poor compliance may result in incorrect outcome of the clinical trial. The term “compliance” refers to the extent to which participants follow the instructions provided to them during the clinical trial. The compliance also includes completion of required trial tasks such as taking medicinal doses and tracking status of the required trial tasks during the clinical trial. Conventionally, the compliance in the clinical trials is managed manually by performing manual tasks such as manually counting investigational product at individual site visit by the participant. Further, the conventional clinical trials may not provide an ability to track compliance in real-time such as validating a timing of dosing. Therefore, the clinical research team may not be able to bring the participants back to compliance. Furthermore, the conventional clinical trials also fail to monitor health condition of the participant during the clinical trial. Thus, the participants may face health risks or adverse effects while participating in the clinical trials.

Hence, there is a need for a system and method for monitoring compliance and participant safety for one or more clinical trials in order to address the aforementioned issues.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In accordance with an embodiment of the present disclosure, a computing system for monitoring compliance and participant safety for one or more clinical trials is disclosed. The computing system includes one or more hardware processors and a memory coupled to the one or more hardware processors. The memory includes a plurality of modules in the form of programmable instructions executable by the one or more hardware processors. The plurality of modules include a trial management module configured to perform one or more clinical trials on a participant for at least one investigational product. The one or more clinical trials includes administering one or more doses of the at least one investigational product to the participant. The plurality of modules also include a health data receiver module configured to receive health data of the participant from one or more data sources after performing the one or more clinical trials on the participant for the at least one investigational product. The health data includes one or more health parameters. The one or more data sources include: one or more Participant Reported Outcome (PRO) parameters. The plurality of modules further include a health monitor module configured to monitor health condition of the participant by analyzing the health data during one or more clinical trial protocols. The one or more clinical trial protocols starts with date of at least one dose intake. Also, the plurality of modules include a health data management module configured to determine whether the received health data meets a predefined safe health condition level using a health condition based Artificial Intelligence (AI) model. The health data management module is also configured to predict adverse effect level of the one or more clinical trials on the participant if the health data fails to meet the predefined safe health condition level. Further, the plurality of modules include a task performer module configured to perform one or more predefined health tasks based on the determined adverse effect level and with respect to the one or more clinical trial protocols to ensure participant safety.

In accordance with another embodiment of the present disclosure, a method for monitoring compliance and participant safety for one or more clinical trials in a computing environment. The method includes performing one or more clinical trials on a participant for at least one investigational product. The one or more clinical trials includes administering one or more doses of the at least one investigational product to the participant. The method also includes receiving health data of the participant from one or more data sources after performing the one or more clinical trials on the participant for the at least one investigational product. The health data includes one or more health parameters. The one or more data sources include: one or more Participant Reported Outcome (PRO) parameters. The method further includes monitoring health condition of the participant by analyzing the health data during one or more clinical trial protocols. The one or more clinical trial protocols start with date of at least one dose intake. Further, the method includes determining whether the received health data meets a predefined safe health condition level using a health condition based Artificial Intelligence (AI) model. Also, the method includes predicting adverse effect level of the one or more clinical trials on the participant if the health data fails to meet the predefined safe health condition level. Furthermore, the method includes performing one or more predefined health tasks based on the predicted adverse effect level and with respect to the one or more clinical trial protocols to ensure participant safety.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF DRAWINGS

The disclosure will be described and explained with additional specificity and detail with the accompanying figures in which:

FIG. 1 is a block diagram illustrating an exemplary computing environment capable of monitoring compliance and participant safety for one or more clinical trials, in accordance with an embodiment of the present disclosure;

FIG. 2 is a process flow diagram illustrating an exemplary method for monitoring compliance and participant safety for one or more clinical trials, in accordance with an embodiment of the present disclosure;

FIGS. 3A-K is a graphical user interface screen of a web application capable of monitoring compliance and participant safety for one or more clinical trials, in accordance with an embodiment of the present disclosure; and

FIGS. 4A-F is a graphical user interface screen of a mobile application capable of monitoring compliance and participant safety for one or more clinical trials, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

Throughout this document, the terms browser and browser application may be used interchangeably to mean the same thing. In some aspects, the terms web application and web app may be used interchangeably to refer to an application, including metadata, that is installed in a browser application. In some aspects, the terms web application and web app may be used interchangeably to refer to a website and/or application to which access is provided over a network (e.g., the Internet) under a specific profile (e.g., a website that provides email service to a user under a specific profile). The terms extension application, web extension, web extension application, extension app and extension may be used interchangeably to refer to a bundle of files that are installed in the browser application to add functionality to the browser application. In some aspects, the term application, when used by itself without modifiers, may be used to refer to, but is not limited to, a web application and/or an extension application that is installed or is to be installed in the browser application.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

Although the explanation is limited to a single participant, it should be understood by the person skilled in the art that the computing system is applied if there are more than one participant.

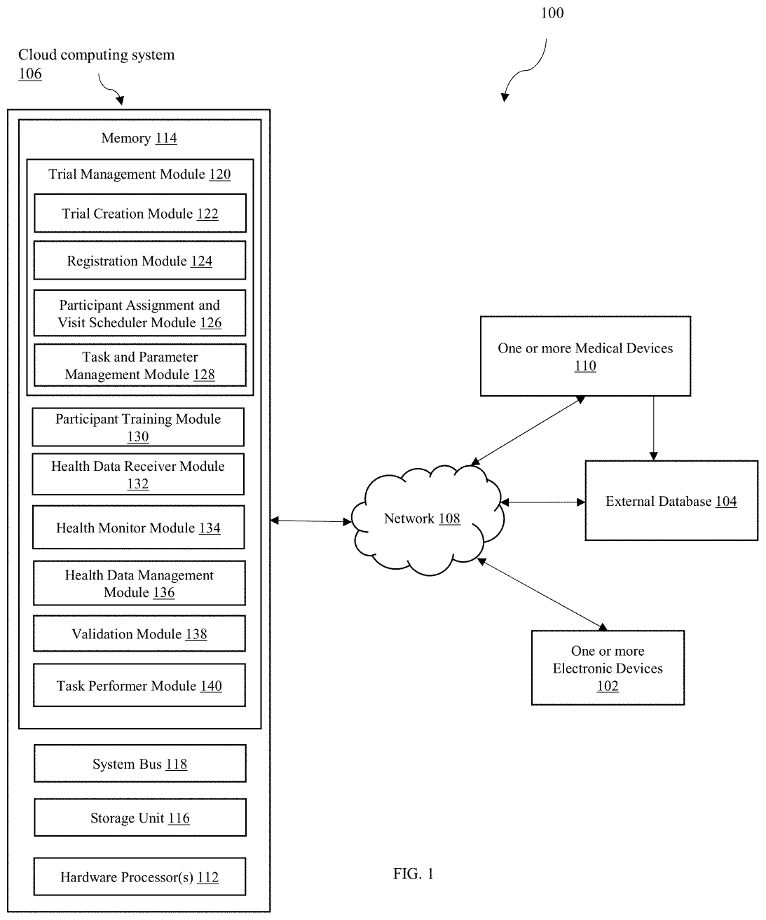

Referring now to the drawings, and more particularly to FIGS. 1 through 4A-F, where similar reference characters denote corresponding features consistently throughout the figures, there are shown preferred embodiments and these embodiments are described in the context of the following exemplary system and/or method.

FIG. 1 is a block diagram illustrating an exemplary computing environment 100 capable of monitoring compliance and participant safety for one or more clinical trials, in accordance with an embodiment of the present disclosure. According to FIG. 1, the computing environment 100 includes one or more electronic devices 102 and an external database 104 communicatively coupled to a computing system 106 via a network 108. In an exemplary embodiment of the present disclosure, the one or more electronic devices 102 include a laptop computer, desktop computer, tablet computer, smartphone, wearable device and smart watch and the like. In an exemplary embodiment of the present disclosure, the network 108 may be internet or any other wireless network. Further, the computing system 106 may be a central server, such as cloud server or a remote server.

The one or more electronic devices 102 include a local browser, a mobile application or a combination thereof. The one or more electronic devices 102 may be associated with all types of users such as a participant, clinical trial coordinator, clinical research team and a registered nurse. Further, each user may use a web application via the local browser, the mobile application or a combination thereof to communicate with the computing system 106. Further, the computing environment 100 include one or more medical devices 110 to capture health data associated with the participant, such that the captured health data may be stored in the external database 104. The health data includes one or more health parameters. In an exemplary embodiment of the present disclosure, the one or more health parameters include temperature, blood pressure, glucose level, pulse rate, weight of the participant and the like. Furthermore, the one or medical devices may send the health data to the one or more electronic devices 102 and the computing system 106 via the network 108. In an embodiment of the present disclosure, the one or more medical devices 110 may also be connected with the one or more electronic devices 102 via other connecting means such as Wireless-Fidelity (Wi-Fi) and Bluetooth. The computing system 106 receives the health data from the one or more data sources such as the one or medical devices, the one or more electronic devices 102, the external database 104 or any combination thereof to allow monitoring compliance and participant health for the one or more clinical trials. In an embodiment of the present disclosure, the one or more data sources also includes one or more Participant Reported Outcomes (PRO) parameters. The one or more PRO parameters include one or more custom queries such as validated quality of life questionnaires that the participant need to respond, frequency with which the one or more participants need to respond to the queries, one or more feedbacks from the participant and the like. In an embodiment of the present disclosure, the one or more custom queries associated with the one or more PRO parameters may be prompted on the one or more electronic devices 102 irrespective of deviation in the health data, such that the participant may provide the one or more PRO parameters to the computing system 106. The participant provides the health data associated with him/her and the one or more PRO parameters to the computing system 106 via the web application, mobile application or a combination thereof.

In an embodiment, the computing system 106 is configured to monitor compliance and participant safety for the one or more clinical trials in the computing environment 100. Further, the one or more clinical trials include a virtual trial, a hybrid trial, a decentralized trial and the like. The computing system 106 include one or more hardware processors 112, a memory 114, and a storage unit 116. The one or more hardware processors 112, the memory 114 and the storage unit 116 is communicatively coupled through a system bus 118 or any similar mechanism. The memory 114 include a plurality of modules in the form of programmable instructions executable by the one or more hardware processors 112. Further, the plurality of modules include a trial management module 120, a trial creation module 122, a registration module 124, a participant assignment and visit scheduler module 126, a task and parameter management module 128, a participant training module 130, a health data receiver module 132, a health monitor module 134, a health data management module 136, a validation module 138 and a task performer module 140.

The one or more hardware processors 112, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 112 may also include embedded controllers, such as generic or programmable logic devices or arrays, application specific integrated circuits, single-chip computers, and the like.

The memory 114 may be non-transitory volatile memory and non-volatile memory. The memory 114 may be coupled for communication with the one or more hardware processors 112, such as being a computer-readable storage medium. The one or more hardware processors 112 may execute machine-readable instructions and/or source code stored in the memory 114. A variety of machine-readable instructions may be stored in and accessed from the memory 114. The memory 114 may include any suitable elements for storing data and machine-readable instructions, such as read only memory, random access memory, erasable programmable read only memory, electrically erasable programmable read only memory, a hard drive, a removable media drive for handling compact disks, digital video disks, diskettes, magnetic tape cartridges, memory cards, and the like. In the present embodiment, the memory 114 includes the plurality of modules stored in the form of machine-readable instructions on any of the above-mentioned storage media and may be in communication with and executed by the one or more hardware processors 112.

The storage unit 116 may be a cloud storage. The storage unit 116 may store the health data. The health data may be accessed by the user from any location via the one or more electronic devices 102 associated with the user. In an embodiment of the present disclosure, the health data stored in the storage unit 116 may be in encrypted format.

The trial management module 120 is configured to perform one or more clinical trials on the participant for at least one investigational product such as pharmaceutical product. In an embodiment of the present disclosure, the one or more clinical trials comprise administering one or more doses of the at least one investigational product to the participant. In performing the one or more clinical trials on the participant for the at least one investigational product, the trial management module 120 comprises the trial creation module 122 configured to create the one or more clinical trials for the at least one investigational product. In an embodiment of the present disclosure, the one or more clinical trials may be distributed and decentralized trials. As used herein, the term ‘distributed and decentralized trials’ may refer to a clinical trial in which traditional research site visits may be replaced by a virtual or remote visit where one or more required tasks may be completed by the participant or an at-home visit by a qualified clinical staff such as a registered nurse and the like.

Further, the trial creation module 122 is also configured to provide one or more clinical trial parameters for the at least one investigational product based on the created one or more clinical trials. The one or more clinical parameters is stored in the storage unit 116. In an exemplary embodiment of the present disclosure, the one or more clinical trial parameters include a phase of the one or more clinical trials, a description of the one or more clinical trials, a start date the one or more clinical trials, an end date the one or more clinical trials, one or more clinical trial milestones, one or more dates of the one or more clinical trial milestones, one or more dosing parameters, one or more vitals parameters, the one or more Participant Reported Outcomes (PRO) parameters, number of cohorts, one or more procedures, one or more visit scheduling parameters and the like. Furthermore, the one or more dosing parameters include an amount of dose, frequency of the dose, an administration type of the dose and the like. In an embodiment of the present disclosure, the dose may be defined as an investigational product which is being tested or used as a reference in the clinical trial. The administration type of the dose include an oral dose, a topical dose, a sub-cutaneous dose, a digital therapeutics (DTX) dose and the like. Further, the one or more vital parameters include the one or more medical devices 110 to be used during the one or more clinical trials, frequency of the vitals collection and vitals monitoring and the like. The one or more medical devices 110 include a blood pressure device, a thermometer and the like. In an embodiment of the present disclosure, the one or more medical devices 110 capture the health data associated with the participant, such that the one or medical devices 110 may send the captured health data to the computing system 106 via the network 108. Furthermore, when the one or more medical devices 110 may not be able to send the captured health data to the computing system 106, the participant may provide the health data associated with him/her to the computing system 106 via the one or more electronic devices 102. The one or more procedures may be a set of tasks to be performed by the participant, such as collecting medical history, collecting weight, drug compliance, randomization, informed consent and the like. In an embodiment of the present disclosure, the cohort is a group of participants who share common traits, illness and characteristics. The one or more visit scheduling parameters may be used to schedule visit of the participant. In an exemplary embodiment of the present disclosure, the one or more visit scheduling parameters include duration of visit, visit type, visit name and the like.

Further, the trial management module 120 further comprises the registration module 124 configured to register the participant for the one or more clinical trials by receiving one or more registration details associated with the participant. In an embodiment of the present disclosure, the participant provides the one or more registration details via the one or more electronic devices 102. The one or more registration details include Person Identifiable Information (PII) and medical history of the participant. The PII may be the information used to identify the participant, such as name and contact information of the participant. Further, the medical history of the participant include participant's medical history, family medical history, past surgical history, allergies, social history and medications the participant is taking or may have recently stopped taking and the like. In an embodiment of the present disclosure, the participant clicks on a participant recruitment hyperlink to access participant recruitment website. Furthermore, the participant provides the one or more registration details through the participant recruitment website. The participant also have to answer trial specific screening questions. The one or more registration details along with answers to the trial specific screening questions may be stored in the storage unit 116. When the participant may not meet the clinical trial criteria, the participant is informed that the participant is not qualified to join the one or more clinical trials. When the participant meets the clinical trial criteria, the clinical trial team is notified about the participant via one or means such as email, SMS and the like. Further, the screening visit is scheduled to complete participant consent and screening activities. Further, when the participant is successfully screened, the clinical trial coordinator sets a flag to register the participant into the one or more clinical trials.

The trial management module 120 also comprises the participant assignment and visit scheduler module 126 configured to assign the participant to the created one or more clinical trials based on the provided one or more clinical trial parameters. In an embodiment of the present disclosure, the participant may be assigned with a participant Identity (ID). In an exemplary embodiment of the present disclosure, the participant ID includes a Two-Dimensional (2D) barcode. The participant is assigned with one or more clinical trial coordinators. The participant assignment and visit scheduler module 126 uses the participant ID, description of the one or more clinical trials such as trial name and trial coordinator, the cohort, randomization flag and randomization date for assigning the participant to the one or more clinical trials. As used herein, the term ‘randomization date’ refers to a first day of the at least one clinical trial assigned to the participant. In an embodiment of the present disclosure, when the randomization flag is set true, the participant assignment and visit scheduler module 126 schedules participant visits for performing the one or more clinical trials on the participant based on the one or more visit scheduling parameters. The participant visits are onsite, virtual, at home or any combination thereof.

The trial management module 120 also comprises the task and parameter management module 128 configured to customize one or more trial tasks for the participant based on the provided one or more clinical trial parameters. In an exemplary embodiment of the present disclosure, the one or more trial tasks include taking the one or more doses of the at least one investigational product, using the one or more medical devices 110, providing one or more feedbacks, the one or more PRO parameters and the like. Further, the task and parameter management module 128 is configured to assign the randomization date for the one or more clinical trials to the participant based on the one or more visit scheduling parameters. The task and parameter management module 128 is also configured to collect data of the one or more clinical trial parameters associated with the participant upon assigning the randomization date. In an exemplary embodiment of the present disclosure, the collected data include dosing data, vital data, PRO data, image data and the like. The collected data of the one or more clinical trials may be stored in the storage unit 116. The dosing data includes one or more dosing parameters required for administering one or more doses of the at least one investigational product to the participant. The one or more dosing parameters include the participant ID, name of the investigational product, dose frequency, time of dose and the like. Furthermore, the vital data includes one or more vital parameters required for capturing vitals of the participant. The one or more vital parameters include the participant ID, name of the vital, vital measurement, time of vitals and the like. The PRO data includes one or more PRO parameters required while providing PRO. The one or more PRO parameters include the participant ID, questionnaire, response to each questionnaire, time of PRO and the like. The image data includes data associated with image captured by the participant such as participant ID and time of capturing the image. In one embodiment, the 2D barcode is used by the at least one of the one or more clinical trial coordinators and the registered nurse to identify the participants during research site visit or at home visit while collecting the data. The 2D barcode is scanned and the participant is identified so that the data captured by the one or more clinical trial coordinators and the registered nurse is automatically associated with a record of the participant. In an embodiment of the present disclosure, the task and parameter management module 128 include a cyclic redundancy check to check the integrity of the collected data.

Further, the task and parameter management module 128 generates one or more notifications associated with the one or more trial tasks. The generated one or more notifications are shared with the participant for completing the one or more trial tasks assigned to the participant. For example, if the participant is required to take a dose, then the one or more notifications is generated and shared to the participant to take the dose. In an embodiment of the present disclosure, the task and parameter management module 128 also notifies the one or more clinical trial coordinators about the one or more doses missed or delayed by the participant, vitals of the participant, the PRO parameters of the participant, vitals missed by the participant, the PRO missed by the participant, dose skipped by the participant and the like. In an exemplary embodiment of the present disclosure, the one or more notifications is generated and shared at or prior to time of dosing, at or prior to time of vitals, at or prior to time of PRO, when one or more doses are not taken within predefined time, when one or more vitals are not captured within predefined time, when PRO is not provided within predefined time and when participant selected skip dose option at the time of dosing. Furthermore, the task and parameter management module 128 monitors the one or more trial tasks to determine if the one or more trial tasks assigned to the participant are completed by the participant. In an embodiment of the present disclosure, the task and parameter management module 128 monitors the one or more trial tasks from the randomization date.

The participant training module 130 is configured to provide training data associated with the one or more trial tasks to the participant for providing an understanding of the one or more trial tasks to the participant. The training data includes an image, an audio, a video and the like. For example, the participant training module 130 provides a video to the participant for instructing the participant to measure the vital statistics of the subject.

The health data receiver module 132 is configured to receive the health data of the participant from the one or more data sources after performing the one or more clinical trials on the participant for the at least one investigational product. Further, the health monitor module 134 is configured to monitor health condition of the participant by analysing the health data during one or more clinical trial protocols. In an embodiment of the present disclosure, the one or more clinical trial protocols start with date of at least one dose intake. The one or more clinical trial protocols describe how the one or more clinical trials may be conducted and ensure safety of the participant and integrity of data collected.

The health data management module 136 is configured to determine whether the health data meets a predefined safe health condition level using a health condition based Artificial Intelligence (AI) model. In an embodiment of the present disclosure, in determining whether the health data meets the predefined safe health condition level using the health condition-based AI model, the health data management module 136 determines whether the received health data deviates from the predefined safe health condition level by comparing the received health data with the predefined safe health condition level prestored in the storage unit 116. The predefined safe health condition level may be participant-specific data. In an exemplary embodiment of the present disclosure, the predefined safe health condition is defined as a baseline, a baseline variance percentage value, a set baseline threshold value or a combination thereof. The predefined safe health condition may be defined corresponding to each of the one or more health parameters. The baseline, the baseline variance percentage value and the set baseline threshold value are set at a time of randomization visit of the participant at the research site. The baseline is information associated with the one or more health parameters of the participant at the randomization date. In an exemplary embodiment of the present disclosure, the baseline may be temperature, blood pressure, glucose level, pulse rate, weight of the participant and the like at the randomization date. The set baseline threshold value is an absolute threshold corresponding to the defined baseline. For example, when the set baseline threshold value for temperature is 103 F and the temperature of the participant is above 103 F, it would exceed the set baseline threshold and the clinical research team and the participant is notified about the exceed in temperature. In another example, the set baseline threshold value for the blood pressure may be systolic 150 mmHg and diastolic 100 mmHg. The baseline variance percentage is a threshold associated with the baseline in percentage form. For example, when the baseline for temperature is 98 F and the baseline variance percentage is 10%, the clinical research team and the participant is notified if the temperature of the participant exceed 98 F+(10% of 98 F) i.e., 107.8 F. In another example, the baseline variance percentage value for Blood Pressure may be 20%. In an embodiment of the present disclosure, when the received health data may deviate from the predefined safe health condition level, the clinical research team and the participant may be notified about the deviation of the health data from the predefined safe health condition level by sending one or more warning messages to the one or more electronic devices 102 associated with the clinical research team and the participant. Further, the health data management module 136 obtains one or more external factors associated with the health condition of the participant by prompting one or more questionnaire using an AI chatbot to the participant and in response receiving data associated with the one or more external factors from the participant. In an exemplary embodiment of the present disclosure, the one or more external factors include temperature, precipitation, food items consumed by the participant and the like. For example, when glucose level of the participant increases due to weather conditions i.e., high temperature, the glucose level may deviate from the predefined safe health condition level. The health data management module 136 identifies that the deviation in glucose level may be because of the weather conditions instead of administering one or more doses of the investigational product. Furthermore, the health data management module 136 generates health condition-based AI model for the participant by correlating the obtained one or more external factors with the received health data and the predefined safe condition level. The generated heath condition-based AI model represents one or more possible root causes for the deviation in the received health data with predefined safe health condition level and a risk score associated with each of the one or more health parameters in the received health data. The health data management module 136 is also configured to determine whether the deviation in the received heath data is due to the administered one or more doses of the at least one investigational product based on the generated health condition-based AI model. When the deviation in the received health data is due to the one or more external factors, the deviation in the received health data may not be due to the administered one or more doses of the at least one investigational product.

Further, the health data management module 136 is configured to predict adverse effect level of the one or more clinical trials on the participant if the received health data fails to meet the predefined safe health condition level. The adverse effect level is threshold level of adverse effects of the one or more clinical trials on the participant. In an exemplary embodiment of the present disclosure, the adverse effects include fever, vomiting, fatigue, fibrillation and the like. In an exemplary embodiment of the present disclosure, the adverse effect level of the participant is extremely critical, critical, serious, fair or stable. In an embodiment of the present disclosure, in predicting adverse effect level of the one or more clinical trials on the participant if the health data fails to meet the predefined safe heath condition level, the health data management module 136 compute compliance metric for the participant. In an embodiment of the present disclosure, the compliance metrics is calculated based on completion of the one or more trial tasks divided by total required trial tasks. The completion of the one or more trial tasks is calculated based on a ratio of dosing, the ratio of vitals and the ratio of PRO in accordance with the one or more clinical trial parameters. In an embodiment of the present disclosure, the completion of the one or more trial tasks is defined as when the one or more trial tasks is completed within a pre-defined time or predefined window. For example, if the participant has a target time of 10 am and the pre-defined window of 2 hours to take a dose, then the participant is required to take the dose between 9 am to 11 am based on the received one or more notifications for taking the dose. The participant is allowed to change a dosing time within the pre-defined window. In an embodiment of the present disclosure, the health data management module 136 includes a hierarchy of compliance. In an exemplary embodiment of the present disclosure, the hierarchy of compliance include a first hierarchy, second hierarchy and a third hierarchy. Further, the first hierarchy include a clinical trial compliance across various research sites. Similarly, the second hierarchy include a site level compliance and the third hierarchy may include a participant level compliance. In an embodiment of the present disclosure, the clinical trial compliance at the first hierarchy includes average of the compliance at the various research sites, the site compliance include average of the participant compliance at the particular site and the participant compliance include average of the compliance related to the dosing, the vitals and the PRO. The health data management module 136 also store data of the one or more participants in the storage unit 116. The data of the one or more participants include information about the one or more doses, information about the one or more vitals and the like. The data of the one or more participants may be aggregated per site and per clinical trial. In an embodiment of the present disclosure, the site may be the research site associated with the participant assigned to the one or more clinical trials. Further, the health data management module 136 may also track quantity of investigational product available with the participant, such that when quantity of the investigational product available with the participant may be low, the health data management module 136 may notify the clinical trial coordinator about low quantity of the investigational product.

Further, the data health management module 136 determines an adverse effect score for the participant based on the computed compliance metric and based on the health condition-based AI model. The health data management module 136 may then predict the adverse effect level of the one or more clinical trials on the participant based on the adverse effect score. The validation module 138 is configured to validate the adverse effect level of the one or more clinical trials on the participant based on the one or more possible root causes of deviation of the health data.

In an embodiment of the present disclosure, the health data management module 136 is further configured to predict a risk of fallout level of the participant based on the computed compliance metric for the participant by using the health condition-based AI model. In an exemplary embodiment of the present disclosure, the health condition-based AI model is a linear regression model. In an embodiment of the present disclosure, the health data management module 136 predicts risk of fallout level of the participant by performing predictive analysis by using the compliance metric and the linear regression model. The risk of fallout level may be a probability of the participant leaving the one or more clinical trials. For example, when the participant is not taking one or more doses of the at least one investigational product as per scheduled time, the risk of fallout level of the participant is high and the participant is likely to leave the one or more clinical trials.

The task performer module 140 is configured to perform one or more predefined heath tasks based on the predicted adverse effect level and with respect to the one or more clinical trial protocols to ensure participant safety. In an embodiment of the present disclosure, in performing the one or more predefined health tasks based on the predicted adverse effect level and with respect to the one or more clinical trial protocols to ensure the participant safety, the task performer module 140 retrieves a predefined plan for the determined adverse effect level of the one or more clinical trials on the participant and the one or more clinical trial protocols by mapping the one or more possible root causes with corresponding prestored root causes in the storage unit 116. Further, the task performer module 140 performs the one or more predefined health tasks based on the retrieved predefined plan for the determined adverse effect level and the one or more clinical trial protocols. In an embodiment of the present disclosure, the data associated with the predefined plan, the prestored root causes and the one or more predefined health tasks may be stored in the storage unit 116. In an exemplary embodiment of the present disclosure, the one or more predefined health tasks include scheduling dose levels, notifying the participant and clinical trial team about the adverse effect level of the participant, changing dose levels, changing dose schedule, skipping one or more doses, prescribing one or more medical products to the participant for ensuring safety of the participant and the like. Furthermore, the task performer module 140 switches from a regular mode to an observant mode upon performing the one or more predefined health tasks. In the observant mode, the health data of the participant is monitored until the health data meets the predefined safe health condition level.

Those of ordinary skilled in the art will appreciate that the hardware depicted in FIG. 1 may vary for particular implementations. For example, other peripheral devices such as an optical disk drive and the like, Local Area Network (LAN), Wide Area Network (WAN), Wireless (e.g., Wi-Fi) adapter, graphics adapter, disk controller, input/output (I/O) adapter also may be used in addition or in place of the hardware depicted. The depicted example is provided for the purpose of explanation only and is not meant to imply architectural limitations with respect to the present disclosure.

Those skilled in the art will recognize that, for simplicity and clarity, the full structure and operation of all data processing systems suitable for use with the present disclosure is not being depicted or described herein. Instead, only so much of a computing system 106 as is unique to the present disclosure or necessary for an understanding of the present disclosure is depicted and described. The remainder of the construction and operation of the computing system 106 may conform to any of the various current implementation and practices known in the art.

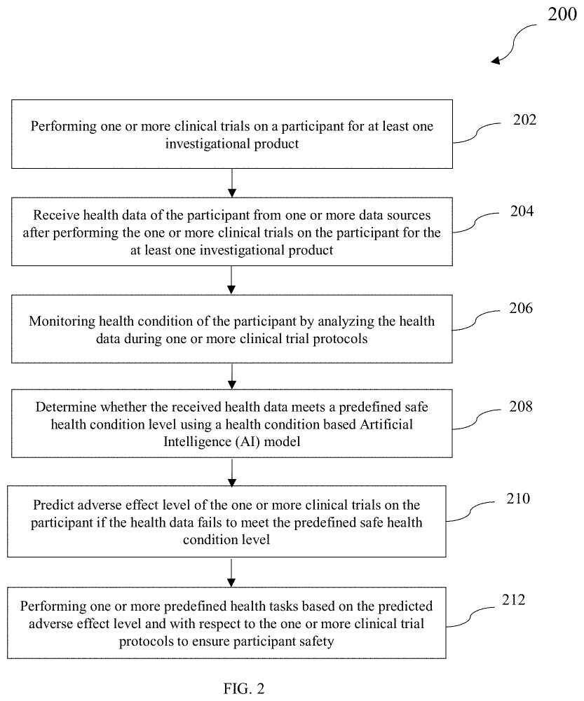

FIG. 2 is a process flow diagram illustrating an exemplary method 200 for monitoring compliance and participant safety for one or more clinical trials in accordance with an embodiment of the present disclosure. At step 202, one or more clinical trials is performed on a participant for at least one investigational product such as investigational product. In an embodiment of the present disclosure, the one or more clinical trials comprise administering one or more doses of the at least one investigational product to the participant. In performing the one or more clinical trials on the participant for the at least one investigational product, the method includes creating the one or more clinical trials for the at least one investigational product. In an embodiment of the present disclosure, the one or more clinical trials may be distributed and decentralized trials. As used herein, the term ‘distributed and decentralized trials may refer to a clinical trial in which traditional research site visits may be replaced by a virtual or remote visit where one or more required tasks may be completed by the participant or an at-home visit by a qualified clinical staff such as a registered nurse and the like.

Further, the method includes providing one or more clinical trial parameters for the at least one investigational product based on the created one or more clinical trials. In an exemplary embodiment of the present disclosure, the one or more clinical trial parameters include a phase of the one or more clinical trials, a description of the one or more clinical trials, a start date the one or more clinical trials, an end date the one or more clinical trials, one or more clinical trial milestones, one or more dates of the one or more clinical trial milestones, one or more dosing parameters, one or more vitals parameters, one or more Participant Reported Outcomes (PRO) parameters, number of cohorts, one or more procedures, one or more visit scheduling parameters and the like. Furthermore, the one or more dosing parameters include an amount of dose, frequency of the dose, an administration type of the dose and the like. In an embodiment of the present disclosure, the dose may be defined as an investigational product which is being tested or used as a reference in the clinical trial. The administration type of the dose includes an oral dose, a topical dose, a sub-cutaneous dose, a digital therapeutics (DTX) dose and the like. In an embodiment of the present disclosure, the participant may be asked to record one or more videos or capture one or more images while taking one or more doses to ensure that the participant is not skipping any dose. Further, the one or more vital parameters include the one or more medical devices 110 to be used during the one or more clinical trials, frequency of the vitals collection and vitals monitoring and the like. The one or more medical devices 110 include a blood pressure device, a thermometer and the like. The one or more medical devices 110 is used to capture health data. In an embodiment of the present disclosure, the health data comprises one or more health parameters. The one or more health parameters include temperature, blood pressure, glucose level, pulse rate, weight of the participant and the like. The one or more PRO parameters include one or more custom queries such as validated quality of life questionnaires that the participant need to respond, frequency with which the participant need to respond to the queries, one or more feedbacks from the participant and the like. The one or more procedures may be a set of tasks to be performed by the participant, such as collecting medical history, collecting weight, drug compliance, randomization, informed consent and the like. In an embodiment of the present disclosure, the cohort is a group of participants who share common traits, illness and characteristics. The one or more visit scheduling parameters may be used to schedule visit of the participant. In an exemplary embodiment of the present disclosure, the one or more visit scheduling parameters include duration of visit, visit type, visit name and the like.

Further, the method includes registering the participant for the one or more clinical trials by receiving one or more registration details associated with the participant. In an embodiment of the present disclosure, the participant provides the one or more registration details via the one or more electronic devices 102. The one or more registration details include Person Identifiable Information (PII) and medical history of the participant. The PII may be the information used to identify the participant, such as name and contact information of the participant. Further, the medical history of the participant includes participant's medical history, family medical history, past surgical history, allergies, social history and medications the participant is taking or may have recently stopped taking and the like. Furthermore, the method includes assigning the participant to the created one or more clinical trials based on the provided one or more clinical trial parameters. In an embodiment of the present disclosure, the participant may be assigned with a participant Identity (ID). In an exemplary embodiment of the present disclosure, the participant ID includes a Two-Dimensional (2D) barcode. The participant is assigned with one or more clinical trial coordinators. The participant ID, description of the one or more clinical trials such as trial name and trial coordinator, the cohort, randomization flag and randomization date are used for assigning the participant to the one or more clinical trials. In an embodiment of the present disclosure, when the randomization flag is set true, participant visits is scheduled for performing the one or more clinical trials on the participant based on the one or more visit scheduling parameters. In an exemplary embodiment of the present disclosure, the participant visit is scheduled at onsite, virtual, at home or any combination thereof.

Furthermore, the method includes customizing one or more trial tasks for the participant based on the provided one or more clinical trial parameters. In an exemplary embodiment of the present disclosure, the one or more trial tasks include taking the one or more doses of the at least one investigational product, using the one or more medical devices 110, providing one or more feedbacks, the one or more PRO parameters and the like. Further, the method includes assigning the randomization date for the one or more clinical trials to the participant based on the one or more visit scheduling parameters. The method also includes collecting data of the one or more clinical trial parameters associated with the participant upon assigning the randomization date. In an exemplary embodiment of the present disclosure, the collected data include dosing data, vital data, PRO data, image data and the like. The dosing data includes one or more dosing parameters required for administering one or more doses of the at least one investigational product to the participant. The one or more dosing parameters include the participant ID, name of the investigational product, dose frequency, time of dose and the like. Furthermore, the vital data includes one or more vital parameters required for capturing vitals of the participant. The one or more vital parameters include the participant ID, name of the vital, vital measurement, time of vitals and the like. The PRO data includes one or more PRO parameters required while providing PRO. The one or more PRO parameters include the participant ID, questionnaire, response to each questionnaire, time of PRO and the like. The image data includes data associated with image captured by the participant such as participant ID and time of capturing the image. In an embodiment of the present disclosure, a cyclic redundancy check is used to check the integrity of the collected data.

Further, the method includes generating one or more notifications associated with the one or more trial tasks. The generated one or more notifications are shared with the participant for completing the one or more trial tasks assigned to the participant. In an embodiment of the present disclosure, the one or more clinical trial coordinators is notified about the one or more doses missed or delayed by the participant, vitals of the participant, the PRO parameters of the participant, vitals missed by the participant, the PRO missed by the participant, dose skipped by the participant and the like. In an exemplary embodiment of the present disclosure, the one or more notifications is generated and shared at or prior to time of dosing, at or prior to time of vitals, at or prior to time of PRO, when one or more doses are not taken within predefined time, when one or more vitals are not captured within predefined time, when PRO is not provided within predefined time and when participant selected skip dose option at the time of dosing. Furthermore, the method includes monitoring the one or more trial tasks to determine if the one or more trial tasks assigned to the participant are completed by the participant. In an embodiment of the present disclosure, the one or more trial tasks are monitored from the randomization date. The method also includes provide training data associated with the one or more trial tasks to the participant for providing an understanding of the one or more trial tasks to the participant. The training data includes an image, an audio, a video and the like.

At step 204, health data of the participant is received from one or more data sources after performing the one or more clinical trials on the participant for the at least one investigational product. In an embodiment of the present disclosure, the one or more data sources includes one or medical devices, one or more electronic devices 102, the external database 104, one or more Participant Reported Outcomes (PRO) parameters or any combination. In an embodiment of the present disclosure, the one or more custom queries associated with the one or more PRO parameters may be prompted on one or more electronic devices 102 irrespective of deviation in the health data, such that the participant may provide the one or more PRO parameters to the computing system 106. The participant may provide the health data associated with him/her and the one or more PRO parameters through the one or more electronic devices 102 via web application, mobile application or a combination thereof. In an exemplary embodiment of the present disclosure, the one or more electronic devices 102 include a laptop computer, desktop computer, tablet computer, smartphone, wearable device and smart watch.

At step 206, health condition of the participant is monitored by analyzing the health data during one or more clinical trial protocols. In an embodiment of the present disclosure, the one or more clinical trial protocols start with date of at least one dose intake. The one or more clinical trial protocols describe how the one or more clinical trials may be conducted and ensure safety of the participant and integrity of data collected.

At step 208, it is determined whether the received health data meets a predefined safe health condition level using a health condition based Artificial Intelligence (AI) model. In determining whether the health data meets the predefined safe health condition level using a health condition-based AI model, the method includes determining whether the received health data deviates from the predefined safe health condition level by comparing the received health data with the predefined safe health condition level prestored in a storage unit 116. The predefined safe health condition level may be participant-specific data. The predefined safe health condition level may be participant-specific data. In an exemplary embodiment of the present disclosure, the predefined safe health condition may be defined as a baseline, a baseline variance percentage value, a set baseline threshold value or a combination thereof. The predefined safe health condition may be defined corresponding to each of the one or more health parameters. The baseline, the baseline variance percentage value and the set baseline threshold value may be set at a time of randomization visit of the participant at the research site. The baseline is information associated with the one or more health parameters of the participant at the randomization date. In an exemplary embodiment of the present disclosure, the baseline may be temperature, blood pressure, glucose level, pulse rate, weight of the participant and the like at the randomization date. The set baseline threshold value is an absolute threshold corresponding to the defined baseline. For example, when the set baseline threshold value for temperature is 103 F and the temperature of the participant is above 103 F, it would exceed the set baseline threshold and the clinical research team and the participant is notified about the exceed in temperature. In another example, the set baseline threshold value for the blood pressure may be systolic 150 mmHg and diastolic 100 mmHg. The baseline variance percentage is a threshold associated with the baseline in percentage form. For example, when the baseline for temperature is 98 F and the baseline variance percentage is 10%, the clinical research team and the participant is notified if the temperature of the participant exceed 98 F+(10% of 98 F) i.e., 107.8 F. In another example, the baseline variance percentage value for Blood Pressure may be 20%. Further, the method includes obtaining one or more external factors associated with the health condition of the participant by prompting one or more questionnaire using an AI chatbot to the participant and in response receiving data associated with the one or more external factors from the participant. In an exemplary embodiment of the present disclosure, the one or more external factors include temperature, precipitation, food items consumed by the participant and the like. Furthermore, the method includes generating health condition-based AI model for the participant by correlating the obtained one or more external factors with the received health data and the predefined safe condition level. The generated heath condition-based AI model represents one or more possible root causes for the deviation in the received health data with predefined safe health condition level and a risk score associated with each of the one or more health parameters in the received health data. The method includes determining whether the deviation in the received heath data is due to the administered one or more doses of the at least one investigational product based on the generated health condition-based AI model. When the deviation in the received health data is due to the one or more external factors, the deviation in the received health data may not be due to the administered one or more doses of the at least one investigational product.

At step 210, adverse effect level of the one or more clinical trials on the participant is predicted if the health data fails to meet the predefined safe health condition level. The adverse effect level is threshold level of adverse effects of the one or more clinical trials on the participant. In an exemplary embodiment of the present disclosure, the adverse effects include fever, vomiting, fatigue, fibrillation and the like. In an exemplary embodiment of the present disclosure, the adverse effect level of the participant is extremely critical, critical, serious, fair or stable. In predicting the adverse effect level of the one or more clinical trials on the participant if the health data fails to meet the predefined safe health condition level, the method includes computing compliance metric for the participant. In an embodiment of the present disclosure, the compliance metrics is calculated based on completion of the one or more trial tasks divided by total required trial tasks. The completion of the one or more trial tasks is calculated based on a ratio of dosing, the ratio of vitals and the ratio of PRO in accordance with the one or more clinical trial parameters. In an embodiment of the present disclosure, the completion of the one or more trial tasks is defined as when the one or more trial tasks is completed within a pre-defined time or predefined window Further, the method includes ranking a hierarchy of compliance such as first may include clinical trial compliance, second may include site compliance and third may include participant compliance. In such embodiment, ranking the hierarchy compliance may include ranking the clinical trial compliance may include average of the site level compliance, the site compliance may include average of the participant level compliance and the participant compliance may include average of dosing, vitals and PRO. Data of the participant may also be stored in the storage unit 116. The data of the participant include information about the one or more doses, information about the one or more vitals and the like. The data of the participant may be aggregated per site and per clinical trial. In an embodiment of the present disclosure, the site may be the research site associated with the participant assigned to the one or more clinical trials. Further, quantity of investigational product available with the participant is also tracked, such that when quantity of the investigational product available with the participant may be low, the clinical trial coordinator is notified about low quantity of the investigational product. The method also includes determining an adverse effect score for the participant based on the computed compliance metric and based on the health condition-based AI model. Further, the method includes predicting the adverse effect level of the one or more clinical trials on the participant based on the adverse effect score. Further, the method includes validating the adverse effect level of the one or more clinical trials on the participant based on the one or more possible root causes of deviation of the health data.

In an embodiment of the present disclosure, the method includes predicting a risk of fallout level of the participant based on the computed compliance metric for the participant by using the health condition-based AI model. In an exemplary embodiment of the present disclosure, the health condition-based AI model is a linear regression model. In an embodiment of the present disclosure, the method includes predicting risk of fallout level of the participant by performing predictive analysis by using the compliance metric and the linear regression model. The risk of fallout level may be a probability of the participant leaving the one or more clinical trials.

At step 212, one or more predefined heath tasks is performed based on the predicted adverse effect level and with respect to the one or more clinical trial protocols to ensure participant safety. In performing the one or more predefined heath tasks based on the predicted adverse effect level and with respect to the one or more clinical trial protocols to ensure the participant safety, the method includes retrieving a predefined plan for the determined adverse effect level of the one or more clinical trials on the participant and the one or more clinical trial protocols by mapping the one or more possible root causes with corresponding prestored root causes in the storage unit 116. Further, the method includes performing the one or more predefined health tasks based on the retrieved predefined plan. In an exemplary embodiment of the present disclosure, the one or more predefined health tasks include scheduling dose levels, notifying the participant and clinical trial team about the adverse effect level of the participant, changing dose levels, changing dose schedule, skipping one or more doses, prescribing one or more medical products to the participant for ensuring safety of the participant and the like. The method also includes switching from a regular mode to an observant mode upon performing the one or more predefined health tasks. In the observant mode, the health data may be monitored until the health data meets the predefined safe health condition level. The method 200 may be implemented in any suitable hardware, software, firmware, or combination thereof.

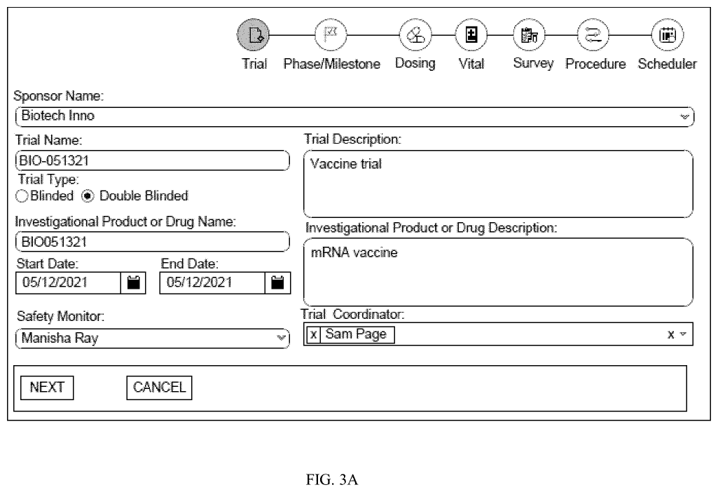

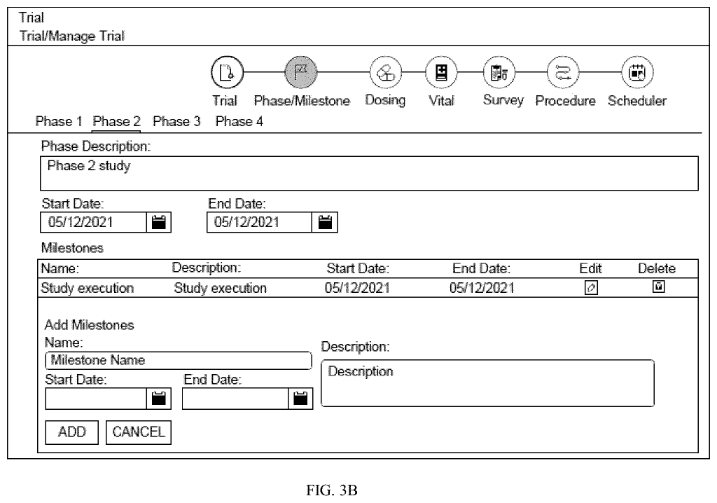

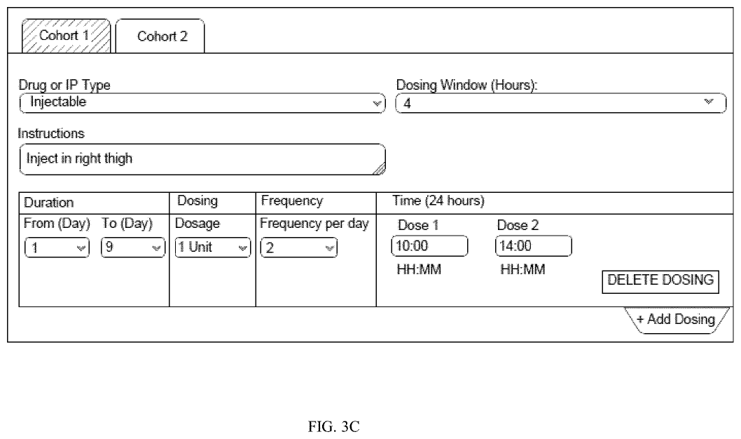

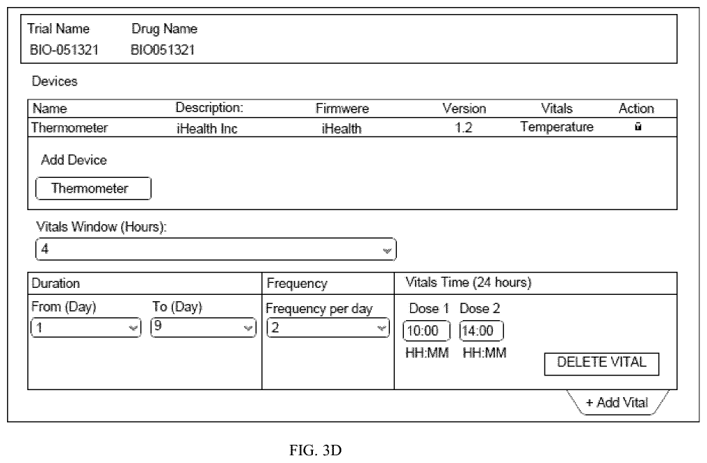

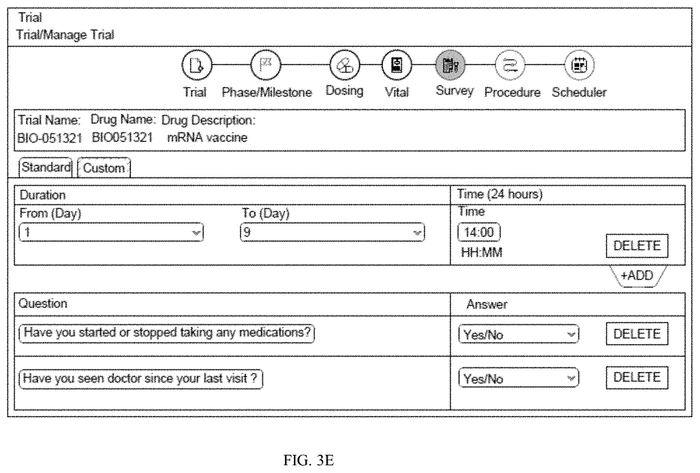

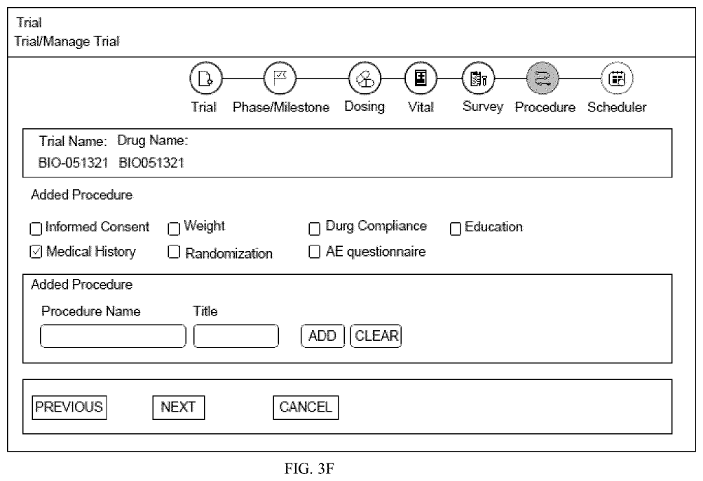

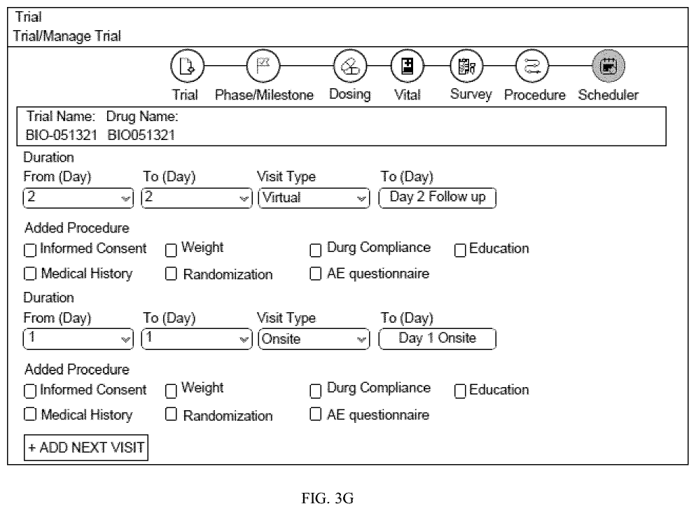

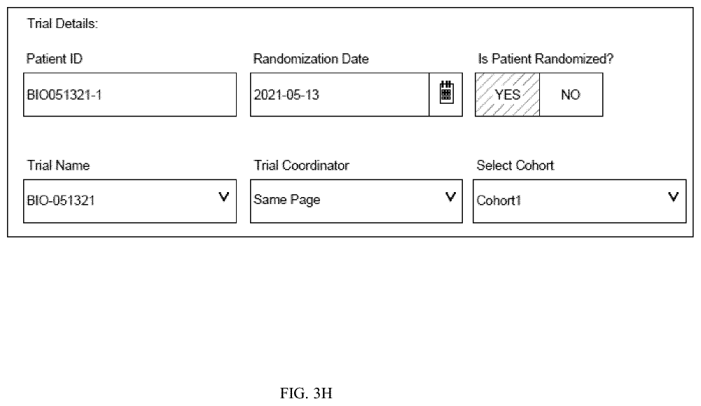

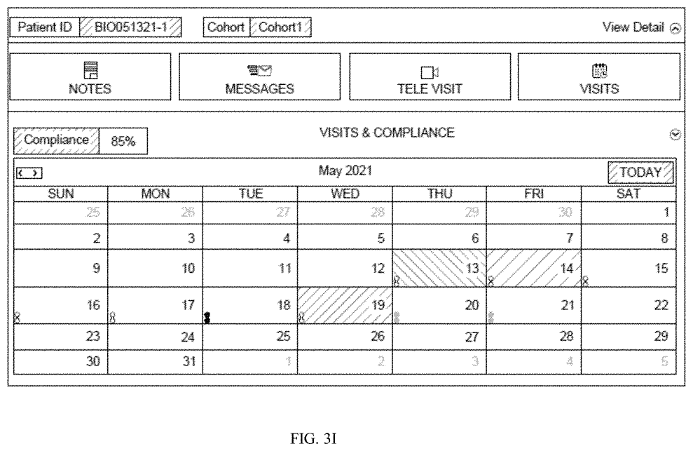

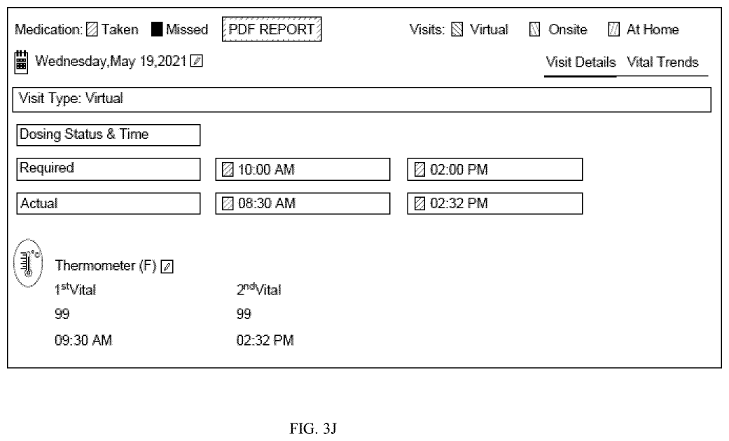

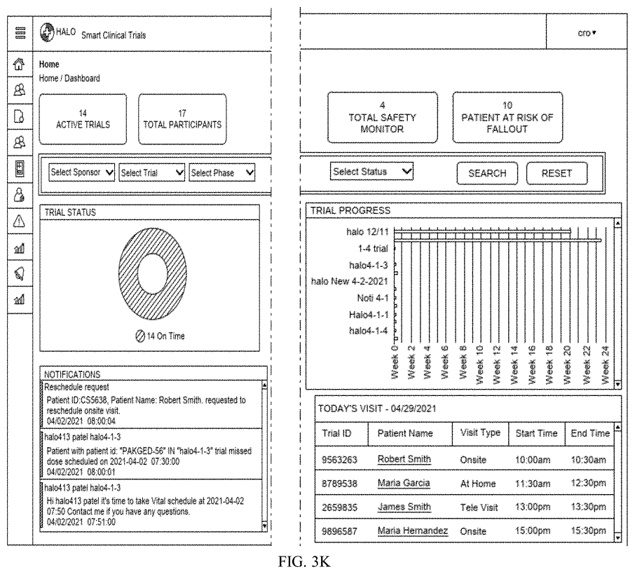

FIGS. 3A-K is a graphical user interface screen of a web application capable of monitoring compliance and participant safety for one or more clinical trials in accordance with an embodiment of the present disclosure. In an embodiment of the present disclosure, the web application is used by the clinical trial coordinator, clinical research team and the registered nurse. FIG. 3A is the graphical user interface screen of the web application which is used to create the one or more trials for the at least one investigational product, which is earlier explained with respect to FIG. 1. In an exemplary embodiment of the present disclosure, the user has to specify sponsor name, trial name, trial description, trial type, start date of the clinical trial, end date of the clinical trial, trial coordinator and the like, as shown in FIG. 3A.

Further, FIG. 3B is the graphical user interface screen of the web application which is used to add phase of the one or more clinical trials. In an exemplary embodiment of the present disclosure, the user has to select phase, specify phase description, start date, end date of the selected phase, name of milestone and the like, as shown in FIG. 3B.

Furthermore, FIG. 3C is the graphical user interface screen of the web application which is used to provide one or more dosing parameters. In an exemplary embodiment of the present disclosure, the user has to add drug type, dosing window, instructions and the like.

FIG. 3D is the graphical user interface screen of the web application which is used to provide one or more vital parameters. In an exemplary embodiment of the present disclosure, the user has to add one or medical devices, specify vital windows, time and frequency of vitals.

Moreover, FIG. 3E is the graphical user interface screen of the web application which is used to perform survey for collecting one or more PRO parameters. In an exemplary embodiment of the present disclosure, the user has to select whether to use standard queries from the storage unit 116 or create custom queries. In an exemplary embodiment of the present disclosure, the user has to specify duration, time and the like, as shown in FIG. 3E. Further, FIG. 3F is the graphical user interface screen of the web application which is used to add one or more procedures. In an exemplary embodiment of the present disclosure, the user may use checkbox to select added procedures, such as informed consent, weight, drug compliance and the like. The user may also use yes and no options to select added procedures. The user may add a new procedure by specifying procedure name and title, as shown in FIG. 3F. FIG. 3G is the graphical user interface screen of the web application which is used to add one or more visit scheduling parameters to schedule visit of the participant based on the one or more clinical trial parameters. For example, when a procedure to collect weight of the participant is added by using graphical user interface screen shown in FIG. 3F, the user may select a day of the trial, such as first day or second day of the trial, for collecting weight of the participant. In an exemplary embodiment of the present disclosure, the user has to specify duration of visit, visit type, visit name and the like. FIG. 3H is the graphical user interface screen of the web application which is used to assign the participant to the created one or more clinical trials based on the one or more clinical trial parameters, which is earlier explained with respect to FIG. 1. In an exemplary embodiment of the present disclosure, the user has to add participant ID, trial name, trial coordinator, cohort, randomization flag, randomization date duration of visit, visit type, visit name and the like. Further, FIG. 3I is the graphical user interface screen of the web application which is accessed by the clinical trial coordinator to view compliance metrics in the form of percentage. FIG. 3J is the graphical user interface screen of the web application which is accessed by the clinical trial coordinator to view vitals of the participant. FIG. 3K is the graphical user interface screen of the web application which is accessed by the clinical trial coordinator to view trial status, trial progress, notifications, participant at risk of fallout and the like.

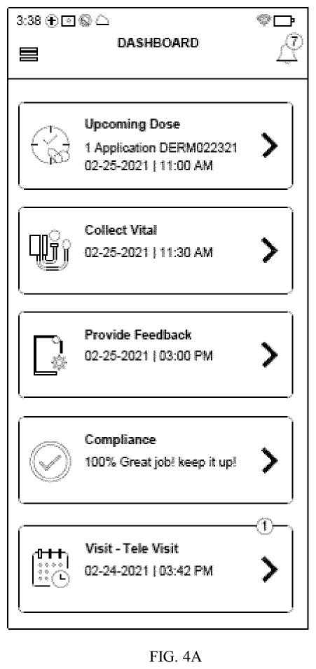

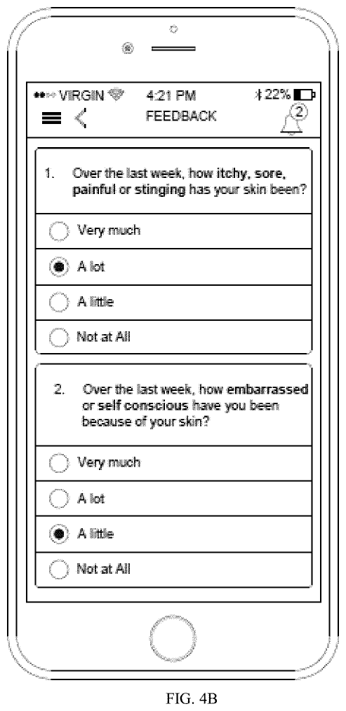

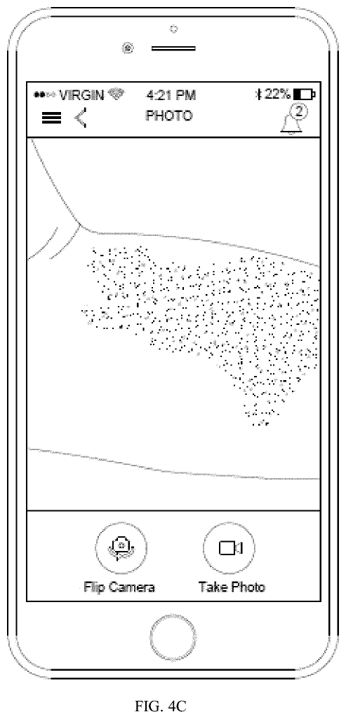

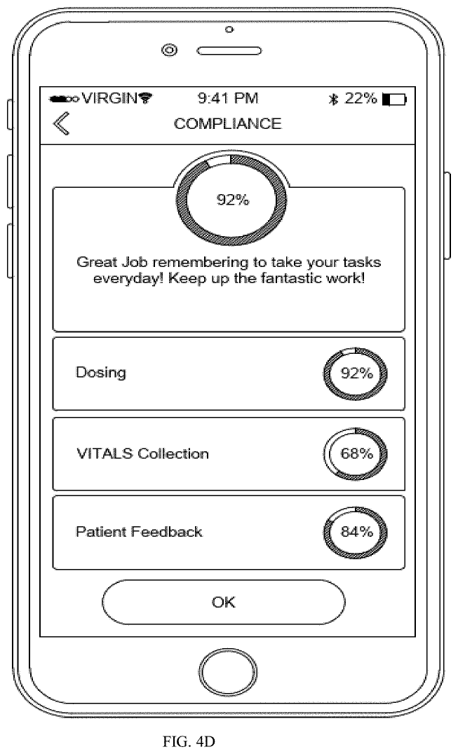

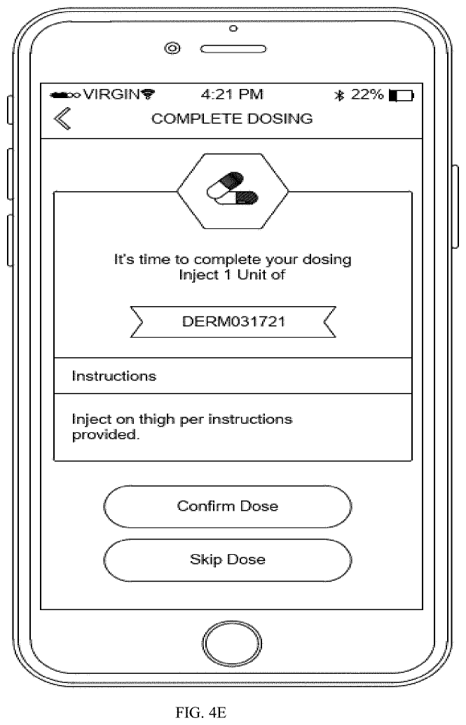

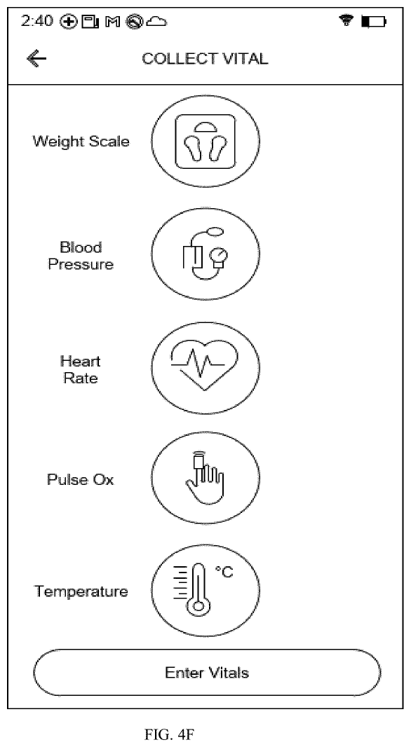

FIGS. 4A-F is a graphical user interface screen of a mobile application capable of monitoring compliance and participant safety for one or more clinical trials in accordance with an embodiment of the present disclosure. In an embodiment of the present disclosure, the mobile application is used by the participant. FIG. 4A is the graphical user interface screen of the mobile application which is used by the user to receive notifications associated with the upcoming dose, collect vital (heath data), provide feedback, view compliance metrics and scheduled visit. Further, FIG. 4B is the graphical user interface screen of the mobile application which is used to provide one or more feedbacks. Furthermore, FIG. 4C is the graphical user interface screen of the mobile application which is used to capture image. In FIG. 4C, the participant has captured an image to consult about an allergy with the registered nurse. FIG. 4D is the graphical user interface screen of the mobile application which is used by the user to view the compliance metrics, compliance for dosing, compliance for vital collection and compliance for participant feedback. This feedback is further updated as a PRO. 4E is the graphical user interface screen of the mobile application which is used by the user to view dosing amount, instructions and options to confirm or skip the dose. 4F is the graphical user interface screen of the mobile application which is used by the user to collect vitals, such as weight, blood pressure, heartrate, pulse Ox, temperature and the like.

Thus, various embodiments of the present computing system 106 provide an efficient solution for monitoring compliance and participant safety for one or more clinical trials. The computing system 106 provides an efficient system to analyze the investigational product such as drug on humans. Moreover, the computing system 106 provides an efficient smart clinical trial which enables a discovery of multiple lifesaving drugs by providing a flexible, online and easy to use platform for clinical trials. Further, the computing system 106 allows monitoring of the one or more trial tasks performed by the participant in the one or more clinical trials. Furthermore, the computing system 106 has various features such as participant compliance, workflow management and the like, which makes the computing system 106 smart and quick without having much manual work. The computing system 106 may also monitor health condition of the participant to determine adverse effect level of the one or more clinical trials on the participant. Further, the computing system 106 also identifies one or more root causes of the deviation of the health data from the predefined safe health condition level. The computing system 106 may also perform one or more predefined health tasks based on the adverse effect level and with respect to the one or more clinical trial protocols to ensure participant safety.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

Input/output (I/O) devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

A representative hardware environment for practicing the embodiments may include a hardware configuration of an information handling/computer system in accordance with the embodiments herein. The system herein comprises at least one processor or central processing unit (CPU). The CPUs are interconnected via system bus 118 to various devices such as a random-access memory (RAM), read-only memory (ROM), and an input/output (I/O) adapter. The I/O adapter can connect to peripheral devices, such as disk units and tape drives, or other program storage devices that are readable by the system. The system can read the inventive instructions on the program storage devices and follow these instructions to execute the methodology of the embodiments herein.

The system further includes a user interface adapter that connects a keyboard, mouse, speaker, microphone, and/or other user interface devices such as a touch screen device (not shown) to the bus to gather user input. Additionally, a communication adapter connects the bus to a data processing network, and a display adapter connects the bus to a display device which may be embodied as an output device such as a monitor, printer, or transmitter, for example.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The specification has described a method and a system for. The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

We claim:

1. A computing system for monitoring compliance and participant safety for one or more clinical trials in a computing environment, the computing system comprising:
one or more hardware processors; and
a memory coupled to the one or more hardware processors, wherein the memory comprises a plurality of modules in the form of programmable instructions executable by the one or more hardware processors, wherein the plurality of modules comprises:
a trial management module configured to perform one or more clinical trials on a participant for at least one investigational product, wherein the one or more clinical trials comprises administering one or more doses of the at least one investigational product to the participant;
a health data receiver module configured to receive health data of the participant from one or more data sources after performing the one or more clinical trials on the participant for the at least one investigational product, wherein the health data comprises one or more health parameters, wherein the one or more data sources comprise: one or more Participant Reported Outcome (PRO) parameters;
a health monitor module configured to monitor health condition of the participant by analyzing the health data during one or more clinical trial protocols, wherein the one or more clinical trial protocols start with date of at least one dose intake;
a health data management module configured to:
determine whether the received health data meets a predefined safe health condition level using a health condition based Artificial Intelligence (AI) model; and
predict adverse effect level of the one or more clinical trials on the participant if the health data fails to meet the predefined safe health condition level; and
a task performer module configured to perform one or more predefined health tasks based on the predicted adverse effect level and with respect to the one or more clinical trial protocols to ensure participant safety.

2. The computing system of claim 1, wherein the task performer module switches from a regular mode to an observant mode upon performing the one or more predefined health tasks, wherein in the observant mode, the health data is monitored until the health data meets the predefined safe health condition level.

3. The computing system of claim 1, wherein in determining whether the received health data meets the predefined safe health condition level using the health condition based Artificial Intelligence (AI) model, the health data management module is configured to:
determine whether the received health data deviates from the predefined safe health condition level by comparing the received health data with the predefined safe health condition level prestored in a storage unit;
obtain one or more external factors associated with the health condition of the participant by prompting one or more questionnaires using an AI chatbot to the participant and in response receiving data associated with the one or more external factors from the participant;
generate health condition-based AI model for the participant by correlating the obtained one or more external factors with the received health data and the predefined safe condition level, wherein the generated heath condition-based AI model represents one or more possible root causes for the deviation in the received health data with predefined safe health condition level and a risk score associated with each of the one or more health parameters in the received health data; and
determine whether the deviation in the received heath data is due to the administered one or more doses of the at least one investigational product based on the generated health condition-based AI model.

4. The computing system of claim 1, wherein in predicting adverse effect level of the one or more clinical trials on the participant if the health data fails to meet the predefined safe health condition level, the health data management module is configured to:
compute compliance metric for the participant, wherein the compliance metrics is calculated based on completion of the one or more trial tasks divided by total required trial tasks;
determine an adverse effect score for the participant based on the computed compliance metric and based on the health condition-based AI model; and
predict the adverse effect level of the one or more clinical trials on the participant based on the adverse effect score.

5. The computing system of claim 4, wherein the health data management module is further configured to predict a risk of fallout level of the participant based on the computed compliance metric for the participant by using the health condition-based AI model.

6. The computing system of claim 1, wherein in performing the one or more predefined health tasks based on the predicted adverse effect level and with respect to the one or more clinical trial protocols to ensure the participant safety, the task performer module is configured to:
retrieve a predefined plan for the determined adverse effect level of the one or more clinical trials on the participant and the one or more clinical trial protocols by mapping the one or more possible root causes with corresponding prestored root causes in a storage unit; and
perform the one or more predefined health tasks based on the retrieved predefined plan for the determined adverse effect level, wherein the one or more predefined health tasks include scheduling dose levels, notifying the participant and clinical trial team about the adverse effect level of the participant, changing dose levels, changing dose schedule, skipping one or more doses and prescribing one or more medical products to the participant for ensuring safety of the participant.

7. The computing system of claim 1, wherein in performing the one or more clinical trials on the participant for the at least one investigational product, the trial management module comprises:
a trial creation module configured to:
create the one or more clinical trials for the at least one investigational product; and
provide one or more clinical trial parameters for the at least one investigational product based on the created one or more clinical trials, wherein the one or more clinical trial parameters comprise: a phase of the one or more clinical trials, a description of the one or more clinical trials, a start date of the one or more clinical trials, an end date of the one or more clinical trials, one or more clinical trial milestones, one or more dates of the one or more clinical trial milestones, one or more dosing parameters, one or more vitals parameters, the one or more Participant Reported Outcomes (PRO) parameters, number of cohorts, one or more procedures and one or more visit scheduling parameters;
a registration module configured to register the participant for the one or more clinical trials by receiving one or more registration details associated with the participant, wherein the one or more registration details comprise: Person Identifiable Information (PII) and medical history of the participant; and
a participant assignment and visit scheduler module configured to:
assign the participant to the created one or more clinical trials based on the provided one or more clinical trial parameters; and
schedule participant visits for performing the one or more clinical trials on the participant based on the one or more visit scheduling parameters, wherein the participant visits are at least one of: onsite, virtual and at home.

8. The computing system of claim 7, wherein the trial management module further comprises a task and parameter management module configured to:
customize one or more trial tasks for the participant based on the provided one or more clinical trial parameters;
assign a randomization date for the one or more clinical trials to the participant based on the one or more visit scheduling parameters;
collect data of the one or more clinical trial parameters associated with the participant upon assigning the randomization date, wherein the collected data comprises: dosing data, vital data, PRO data and image data;
generate one or more notifications associated with the one or more trial tasks; wherein the generated one or more notifications are shared with the participant for completing the one or more trial tasks assigned to the participant; and
monitor the one or more trial tasks to determine if the one or more trial tasks assigned to the participant are completed by the participant.

9. A method for monitoring compliance and participant safety for one or more clinical trials in a computing environment, the method comprising:
performing, by one or more hardware processors, one or more clinical trials on a participant for at least one investigational product, wherein the one or more clinical trials comprises administering one or more doses of the at least one investigational product to the participant;
receiving, by the one or more hardware processors, health data of the participant from one or more data sources after performing the one or more clinical trials on the participant for the at least one investigational product, wherein the health data comprises one or more health parameters, wherein the one or more data sources comprise: one or more Participant Reported Outcome (PRO) parameters;
monitoring, by the one or more hardware processors, health condition of the participant by analyzing the health data during one or more clinical trial protocols, wherein the one or more clinical trial protocols start with date of at least one dose intake;
determining, by the one or more hardware processors, whether the received health data meets a predefined safe health condition level using a health condition based Artificial Intelligence (AI) model;
predicting, by the one or more hardware processors, adverse effect level of the one or more clinical trials on the participant if the health data fails to meet the predefined safe health condition level; and
performing, by the one or more hardware processors, one or more predefined health tasks based on the predicted adverse effect level and with respect to the one or more clinical trial protocols to ensure participant safety.

10. The method of claim 9, further comprises switching from a regular mode to an observant mode upon performing the one or more predefined health tasks, wherein in the observant mode, the health data is monitored until the health data meets the predefined safe health condition level.

11. The method of claim 9, wherein determining whether the health data meets the predefined safe health condition level using the health condition-based AI model comprises:
determining whether the received health data deviates from the predefined safe health condition level by comparing the received health data with the predefined safe health condition level prestored in a storage unit;
obtaining one or more external factors associated with the health condition of the participant by prompting one or more questionnaires using an AI chatbot to the participant and in response receiving data associated with the one or more external factors from the participant;
generating health condition-based AI model for the participant by correlating the obtained one or more external factors with the received health data and the predefined safe condition level, wherein the generated heath condition-based AI model represents one or more possible root causes for the deviation in the received health data with predefined safe health condition level and a risk score associated with each of the one or more health parameters in the received health data; and
determining whether the deviation in the received heath data is due to the administered one or more doses of the at least one investigational product based on the generated health condition-based AI model.

12. The method of claim 9, wherein predicting adverse effect level of the one or more clinical trials on the participant if the health data fails to meet the predefined safe health condition level comprises:
computing compliance metric for the participant, wherein the compliance metrics is calculated based on completion of the one or more trial tasks divided by total required trial tasks;
determining an adverse effect score for the participant based on the computed compliance metric and based on the health condition-based AI model; and
predicting the adverse effect level of the one or more clinical trials on the participant based on the adverse effect score.

13. The method of claim 12, further comprises predicting a risk of fallout level of the participant based on the computed compliance metric for the participant by using the health condition-based AI model.

14. The method of claim 9, wherein performing the one or more predefined health tasks based on the predicted adverse effect level and with respect to the one or more clinical trial protocols to ensure participant safety comprises:
retrieving a predefined plan for the determined adverse effect level of the one or more clinical trials on the participant and the one or more clinical trial protocols by mapping the one or more possible root causes with corresponding prestored root causes in a storage unit; and
performing the one or more predefined health tasks based on the retrieved predefined plan for the determined adverse effect level, wherein the one or more predefined health tasks include scheduling dose levels, notifying the participant and clinical trial team about the adverse effect level of the participant, changing dose levels, changing dose schedule, skipping one or more doses and prescribing one or more medical products to the participant for ensuring safety of the participant.

15. The method of claim 9, wherein performing the one or more clinical trials on the participant for the at least one investigational product comprises:
creating the one or more clinical trials for the at least one investigational product;
providing one or more clinical trial parameters for the at least one investigational product based on the created one or more clinical trials, wherein the one or more clinical trial parameters comprise: a phase of the one or more clinical trials, a description of the one or more clinical trials, a start date of the one or more clinical trials, an end date of the one or more clinical trials, one or more clinical trial milestones, one or more dates of the one or more clinical trial milestones, one or more dosing parameters, one or more vitals parameters, the one or more Participant Reported Outcomes (PRO) parameters, number of cohorts, one or more procedures and one or more visit scheduling parameters;
registering the participant for the one or more clinical trials by receiving one or more registration details associated with the participant, wherein the one or more registration details comprise: Person Identifiable Information (PII) and medical history of the participant;
assigning the participant to the created one or more clinical trials based on the provided one or more clinical trial parameters;
scheduling participant visits for performing the one or more clinical trials on the participant based on the one or more visit scheduling parameters, wherein the participant visits are at least one of: onsite, virtual and at home.

16. The method of claim 15, further comprises:
customizing one or more trial tasks for the participant based on the provided one or more clinical trial parameters;
assigning a randomization date for the one or more clinical trials to the participant based on the one or more visit scheduling parameters;
collecting data of the one or more clinical trial parameters associated with the participant upon assigning the randomization date, wherein the collected data comprises: dosing data, vital data, PRO data and image data;
generating one or more notifications associated with the one or more trial tasks, wherein the generated one or more notifications are shared with the participant for completing the one or more trial tasks assigned to the participant; and
monitoring the one or more trial tasks to determine if the one or more trial tasks assigned to the participant are completed by the participant.

17. A non-transitory computer-readable storage medium having instructions stored therein that, when executed by a hardware processor, cause the processor to perform method steps comprising:
performing one or more clinical trials on a participant for at least one investigational product, wherein the one or more clinical trials comprises administering one or more doses of the at least one investigational product to the participant;
receiving health data of the participant from one or more data sources after performing the one or more clinical trials on the participant for the at least one investigational product, wherein the health data comprises one or more health parameters, wherein the one or more data sources comprise: one or more Participant Reported Outcome (PRO) parameters;
monitoring health condition of the participant by analyzing the health data during one or more clinical trial protocols, wherein the one or more clinical trial protocols start with date of at least one dose intake;
determining whether the received health data meets a predefined safe health condition level using a health condition based Artificial Intelligence (AI) model;
predicting adverse effect level of the one or more clinical trials on the participant if the health data fails to meet the predefined safe health condition level; and
performing one or more predefined health tasks based on the predicted adverse effect level and with respect to the one or more clinical trial protocols to ensure participant safety.

18. The non-transitory computer-readable storage medium of claim 15, further comprises switching from a regular mode to an observant mode upon performing the one or more predefined health tasks, wherein in the observant mode, the health data is monitored until the health data meets the predefined safe health condition level.

19. The non-transitory computer-readable storage medium of claim 15, wherein determining whether the health data meets the predefined safe health condition level using the health condition-based AI model comprises:
determining whether the received health data deviates from the predefined safe health condition level by comparing the received health data with the predefined safe health condition level prestored in a storage unit;
obtaining one or more external factors associated with the health condition of the participant by prompting one or more questionnaires using an AI chatbot to the participant and in response receiving data associated with the one or more external factors from the participant;
generating health condition-based AI model for the participant by correlating the obtained one or more external factors with the received health data and the predefined safe condition level, wherein the generated heath condition-based AI model represents one or more possible root causes for the deviation in the received health data with predefined safe health condition level and a risk score associated with each of the one or more health parameters in the received health data; and
determining whether the deviation in the received heath data is due to the administered one or more doses of the at least one investigational product based on the generated health condition-based AI model.

20. The non-transitory computer-readable storage medium of claim 15, wherein predicting adverse effect level of the one or more clinical trials on the participant if the health data fails to meet the predefined safe health condition level comprises:
computing compliance metric for the participant, wherein the compliance metrics is calculated based on completion of the one or more trial tasks divided by total required trial tasks;
determining an adverse effect score for the participant based on the computed compliance metric and based on the health condition-based AI model; and
predicting the adverse effect level of the one or more clinical trials on the participant based on the adverse effect score.

21. The non-transitory computer-readable storage medium of claim 15, wherein performing the one or more predefined health tasks based on the predicted adverse effect level and with respect to the one or more clinical trial protocols to ensure the participant safety comprises:
retrieving a predefined plan for the determined adverse effect level of the one or more clinical trials on the participant and the one or more clinical trial protocols by mapping the one or more possible root causes with corresponding prestored root causes in a storage unit; and
performing the one or more predefined health tasks based on the retrieved predefined plan for the determined adverse effect level, wherein the one or more predefined health tasks include scheduling dose levels, notifying the participant and clinical trial team about the adverse effect level of the participant, changing dose levels, changing dose schedule, skipping one or more doses and prescribing one or more medical products to the participant for ensuring safety of the participant.

22. The non-transitory computer-readable storage medium of claim 15, wherein performing the one or more clinical trials on the participant for the at least one investigational product comprises:
creating the one or more clinical trials for the at least one investigational product;
providing one or more clinical trial parameters for the at least one investigational product based on the created one or more clinical trials, wherein the one or more clinical trial parameters comprise: a phase of the one or more clinical trials, a description of the one or more clinical trials, a start date of the one or more clinical trials, an end date of the one or more clinical trials, one or more clinical trial milestones, one or more dates of the one or more clinical trial milestones, one or more dosing parameters, one or more vitals parameters, the one or more Participant Reported Outcomes (PRO) parameters, number of cohorts, one or more procedures and one or more visit scheduling parameters;
registering the participant for the one or more clinical trials by receiving one or more registration details associated with the participant, wherein the one or more registration details comprise: Person Identifiable Information (PII) and medical history of the participant;
assigning the participant to the created one or more clinical trials based on the provided one or more clinical trial parameters;
scheduling participant visits for performing the one or more clinical trials on the participant based on the one or more visit scheduling parameters, wherein the participant visits are at least one of: onsite, virtual and at home.
